(19) United States
(12) Patent Application Publication
(10) Pub. No.: US 2008/0306141 A1
(43) Pub. Date: Dec. 11, 2008

(54) Method of Extraction of Catechin Type-A Proanthocyanidins

(75) Inventor: Jan Glinski, Danbury, CT (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Jan Glinski, Danbury, CT (US)

(21) Appl. No.: 11/971,994

(22) Filed: Jan. 10, 2008

Related U.S. Application Data
(60) Provisional application No. 60/884,277, filed on Jan. 10, 2007.

(51) Int. Cl. A61K 31/352; C07D 311/62

(52) U.S. Cl. 514/456; 549/399

(57) ABSTRACT

Catechin type-A proanthocyanidins are recovered from cinnamon bark or other sources rich in catechin type-A proanthocyanidins by extraction with a first organic solvent to provide an extract rich in catechin type-A proanthocyanidins. This extract is combined with water and the organic solvent is removed by distillation so that proanthocyanidins are transferred to the aqueous phase with the precipitation of hydrophobic materials. The aqueous extract is further purified by contact with a second organic solvent that is immiscible with water and appropriate polarity to remove reddish colored, higher polymeric proanthocyanidins from the aqueous phase. The purified aqueous extract is then subjected to evaporative concentration to produce a first solid extract. Extraction of the first solid extract with dry acetone produces an organic extract which, upon removal of the acetone, produces a solid which is enriched in catechin type-A proanthocyanidins. Purity is increased by about another 20%. This purified extract is water-soluble and is free, or essentially free, of typical cinnamon aroma compounds.

### FIELD OF THE INVENTION

A method for obtaining an extract, which is highly enriched into oligophenolic compounds, known as catechin type-A proanthocyanidins, from cinnamon bark and other sources of catechin type-A proanthocyanidins.

### BACKGROUND OF THE INVENTION

Catechin type-A proanthocyanidins distinguish themselves from other proanthocyanidins in that they have two covalent bonds joining adjacent monomeric units. The monomeric units have either catechin or epicatechin carbon skeleton.

Catechin type-A proanthocyanidins extracted from cinnamon may have activity as insulin stimulating compounds that may be useful in the treatment of type 2 diabetes mellitus. See, Anderson et al., “Isolation and Characterization of Polyphenol Type-A Polymers from Cinnamon with Insulin-like Biological Activity”, J. Agric. Food Chem., 52, 65-70 (2004), the disclosure of which is herein incorporated by reference. The extraction process described in the Anderson et al. article includes contact with water containing acetic acid under autoclaving conditions followed by ethanol extraction of the of the supernatant liquid and acetonitrile/acetic acid purification.

Other processes have been disclosed in the art for treating cinnamon. Gourdin et al., U.S. Pat. No. 6,960,360 teaches the use of a solid adsorbent of brominated polystyrene resin that adsorbs total phenols. It is known and generally accepted that the use of a solid phase for adsorption may be associated with such undesired problems as decreased recovery of the adsorbed substances or partial degradation (e.g. formation of polymeric phenols from oligomeric phenols).

As described in U.S. Pat. No. 6,126,940 the extraction of proanthocyanidin from dry plants can be performed wherein the raw material is ground into powder or cut into fine pieces and is extracted batch-wise or continuously with a solvent. The extraction solvent may be a hydrophilic or oleophilic solvent, including, for example water or polar organic or moderately polar organic solvent, including, for example, alcohols such as ethanol, methanol, isopropyl alcohol, etc., ketones such as acetone, methyl ethyl ketone, etc., esters such as methyl acetate, ethyl acetate, etc. These can be used singly or as mixed solvents. The extraction temperature is generally between 0° C. and 100° C. Filtration or centrifugation removes insoluble residues. Purification uses a two-phase solvent partitioning method, a chromatographic column method and a high-performance liquid chromatographic method and the like, singly or as combined. The two-phase solvent partitioning method removes the oil-soluble components and dyes with n-hexane, petroleum ether and partitions the extract into a solvent phase of n-butanol, methyl ethyl ketone. The proanthocyanidin is collected from the solvent phase. As a result of this purification, water-soluble ionic substances, non-ionic substances such as saccharides, polysaccharides, oils, and dyes are removed from the crude extract, and proanthocyanidin is obtained.

U.S. Pat. No. 6,720,353 describes a method of preparing a proanthocyanidin extract from a Vaccinium species which comprises:
(a) homogenizing Vaccinium plant material in an aqueous extraction solvent comprising 10-30% water, 10-70% acetone, 5-60% methanol and 0.05-1% ascorbic acid to prepare a first extract;
(b) clarifying and obtaining the supernatant from the first extract;
(c) removing the solvent from the supernatant and re-suspending the residue in distilled water;
(d) subjecting the re-suspended residue solution to further purification by either:
   i) applying the residue solution to reverse-phase lipophilic chromatography material equilibrated in distilled water and successively washing the lipophilic chromatography material with distilled water to remove sugars, with about 15% aqueous methanol to remove acids and with about 100% acidified methanol to elute polyphenolic compounds, and removing solvent from the polyphenolic fraction to obtain a first dried polyphenolic fraction, or
   (ii) extracting the residue solution with a non-polar extraction solvent such as petroleum ether or hexane, recovering the aqueous phase and removing solvent therefrom to obtain a second dried fraction;
(e) suspending the first or second dried fraction in about 50% aqueous ethanol, applying that solution to mixed hydrophilic-lipophilic chromatography material equilibrated in about 50% aqueous ethanol, and washing the mixed hydrophilic-lipophilic chromatography material with about 50% aqueous ethanol to remove non-proanthocyanidin polyphenolic compounds; and
(f) eluting the mixed hydrophilic-lipophilic chromatography material with about 70% aqueous acetone to obtain the proanthocyanidin extract.

Published US Patent Application No. 20060275515 teaches a cinnamon bark extraction process that includes:
(i) grinding cinnamon bark into powder and stirring it into an aqueous buffer to obtain a solution;
(ii) centrifuging the solution and separating a supernatant,
(iii) introducing a salt to obtain a precipitate,
(iv) dissolving the precipitate obtained in step (iii) above in water or buffer at an essentially neutral pH;
(v) separating the solution on a sepharose or Sephadex column; and
(vi) eluting the solution with suitable buffer and varying concentrations of saccharide, preferably galactose to obtain the antiviral fractions of cinnamon sp.

It would be desirable to have an extraction process for cinnamon bark and other sources of catechin type-A proanthocyanidins that would result in high yield of the desired oligomeric catechin type-A proanthocyanidins without undesired odor or aroma components.

### SUMMARY OF THE INVENTION

It is an object of the invention to provide a process for obtaining an extract highly enriched in the oligomeric catechin type-A proanthocyanidins from a source thereof that is devoid of the components responsible for aroma.

It is a further object of the invention to provide a fast and practical process that does not use harsh chemicals, high temperatures or solid adsorbents.

In accordance with these and other objectives of the invention that will become apparent from the description herein, a preferred process for extracting oligomeric catechin type-A proanthocyanidins from a source thereof includes the steps:
a. contacting a source of catechin type-A proanthocyanidins under extraction conditions with a first organic solvent, preferably acetone or other suitable solvent or solvent mixture, to form a first extract and spent solids;
b. optionally, concentrating the first extract to an elevated concentration of dissolved solids;
c. combining the first extract (or concentrated first extract) with a volume of water, preferably about two to four times larger than the volume of the first extract, to form an aqueous-organic solvent solution;
d. removing organic solvent from the aqueous-organic solvent solution, preferably by evaporation or distillation, to form a first aqueous solution containing soluble proanthocyanidins and to separate at least a portion of the hydrophobic components from said first aqueous solution in the form of oil droplets, oily clumps, or other solids;
e. in one or more stages, washing the first aqueous solution with a second organic solvent, preferably of a different composition than the first organic solvent and comprising or consisting essentially of hexane and ethyl acetate or other solvent that dissolves residual hydrophobic components present in the first aqueous solution (including the components responsible for the characteristic aroma of cinnamon bark), under conditions sufficient to remove additional water insoluble components and form a second aqueous solution that is preferably clear in appearance; and
f. exposing the second aqueous solution to evaporation conditions (preferably in vacuo) until a solid product containing oligomeric proanthocyanidins is obtained.

Further enhancement of the concentration of proanthocyanidins can be obtained by treating the solid with a small volume of a third organic solvent, such as dry acetone or similar ketone or ester solvent. Proanthocyanidins dissolve in this third organic solvent, while about 20% w/w of the initial weight remains undissolved and is discarded. The third organic solution may or may not require filtration or centrifugation for separating solids from the liquid. Evaporation of the third organic solution produces the final product containing oligomeric proantohocyanidins as well as polymeric proanthocyanidins.

The method of the invention involves process steps that eliminate substantially, if not all, of many undesired components from the desired proanthocyanidin extract. The entire sequence utilizes conditions that are exceptionally mild. All steps can be done at the temperatures of, or close to, ambient temperature thereby avoiding the potentially harmful and degrading effects of elevated temperature on oligomeric catechin type-A proanthocyanidins. In addition, the process of the invention does not use solid phase adsorbents and the drawbacks commonly associated with the use of them, e.g., irreversible adsorption, increased polymerization, chemical degradation, etc. The entire process is exceptionally fast, which is an economic advantage. The end product of the invented process affords a fraction of the cinnamon extract that is soluble in water and is devoid of an aroma compounds typically occurring in cinnamon. It contains the water-soluble proanthocyanidins accompanied by polymeric polyphenols that were originally present in the source material.

The extract from the process described herein can be used in a wide variety of preparations including orally or externally applied compositions, pharmaceuticals, therapies, foods and food supplements, cosmetics, personal care compositions, toothpastes, shampoos, and the like.

### BRIEF DESCRIPTION OF THE FIGURE

FIG. 1 is a block diagram illustrating a preferred process of the present invention.

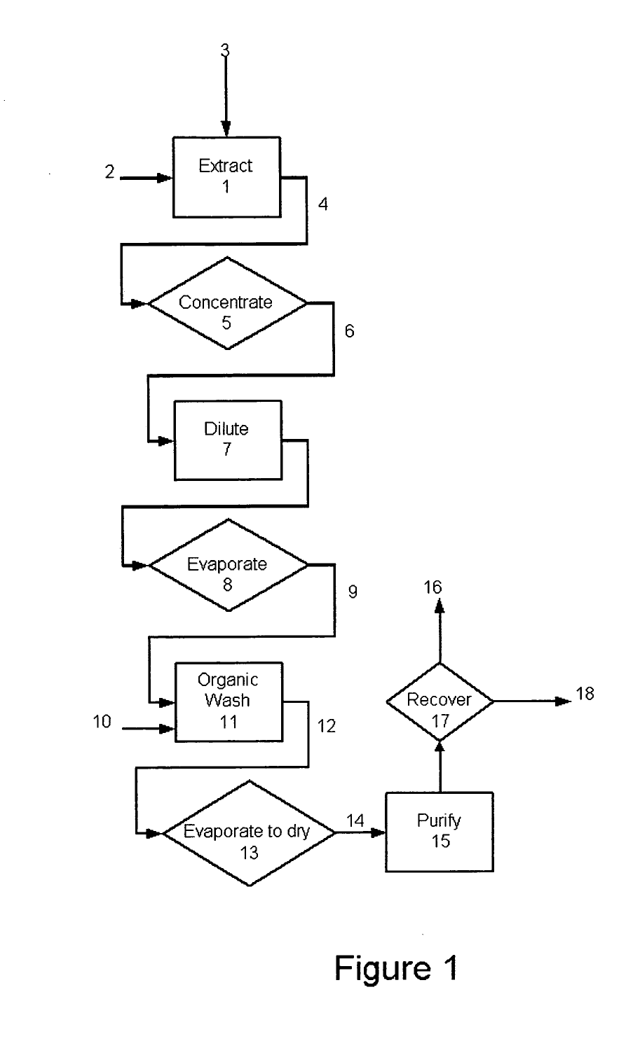

### DETAILED DESCRIPTION OF THE INVENTION

The present invention uses a series of extraction, separation and concentration steps to produce a first extract product that is rich in catechin type-A proanthocyanidins. The extraction process can occur batch-wise or in continuous operations, depending on the type of equipment that is available. Small scale operations may find it convenient to perform the extraction in batch mode.

One or more extraction operations 1 with a first organic solvent 2 can be performed on the feed material 3 until completely spent or a diminishing rate of extraction is seen. Ketone or ester solvents are preferred as the first organic solvent, with acetone being particularly preferred for the process of the present invention.

The preferred source of catechin type-A proanthocyanidins for use in the present invention is cinnamon bark. Those in the art will understand and appreciate that other sources of catechin type-A proanthocyanidins can be used in the present process. Exemplary materials include dried and living cranberries or similar species of plants that include catechin type-A proanthocyanidins.

First extract 4, whether or not made of a combination of individual extracts from the original source solids, is optionally and preferably concentrated to a dissolved solids content of about 1-99 wt %, preferably within a range of about 10-30 wt %. Distillation and/or evaporation can be used in concentrator 5.

Concentrated first extract 6 is combined with 200-400 vol % water in dilution step 7 to form a liquid-liquid solution and subjected to further distillation and/or evaporation 8 under conditions sufficient to remove organic solvent from the concentrated first extract and form a first aqueous solution. Suitable conditions include ambient pressures at a temperature within the range of 20° C. to about 90° C., or reduced pressures at a temperature within the range from about 20° C. to about 80° C. When the organic solvent is removed from the liquid-liquid solution, hydrophobic components of the first extract fall from solution in the form oil droplets, oily clumps, or solid precipitates under low shear conditions.

The first aqueous solution 9 is then combined with a second organic solvent 10 to allow the hydrophobic components, including the fractions responsible for the characteristic cinnamon aroma, to dissolve therein. The organic phase is then separated and discarded. What remains is a second aqueous solution 12 that is preferably at least substantially clear in appearance but which contains dissolved proanthocyanidins. If desired, a second contact with the second organic solvent (not shown) or a variation by ratio, can be used for further purification of the second aqueous solution.

The preferred second organic solvents include a combination of a C5-C8 alkane and a C1-C6 alkyl acetate, propionate or butyrate. Preferably, the second organic solvent is a combination of hexane and ethyl acetate which are, even more preferably, in a weight ratio of 1 part hexane to about 0.1 to 10 parts of ethyl acetate. Chloroform, methylene chloride, or other chlorinated solvents can be used alone or in combination with the other solvents described herein as the second organic solvent.

The proanthocyanidin-containing second aqueous solution 12 is then concentrated in evaporator 13 to a first substantially dry solid product 14. Vacuum conditions may, or may not, be used. The use of vacuum for drying is preferred.

Further purification of the first dry solid proanthocyanidin product 14 can be effected by contacting the first dry solid proanthocyanidin product with a third organic solvent, such as dry acetone, in purifier 15. The desired oligomeric proanthocyanidins dissolve in the third organic solvent, while other components remain insoluble and may be removed by filtration or centrifugation from solution. When third organic solvent 16 is removed in recovery stage 17, second dry solid proanthocyanidin 18 is recovered.

### EXAMPLES

The following example illustrates a preferred form of the present invention. All conclusions are based on the analysis of each step of the process using high performance liquid chromatography equipped with diode array detector and evaporative light scattering detector.

### Example 1

Step 1: Ground or powdered cinnamon bark (Cinnamommum spp. or Cassia spp.) is extracted preferably with acetone or other organic solvents or mixtures of solvents of similar polarity. This step can be carried out preferably at a temperature within the range of about 0° to below the boiling point of the organic solvent, e.g., about 63°-65° C. but temperatures within the range from about 10° to about 50° C. are also permissible. The use of autoclaving or applied pressures can permit the use of higher temperatures, e.g., up to about 75°-90° C.

Sequential extraction of 500 grams of powdered bark with 2.5 L of acetone and then with another 2 liters of acetone with an intermittent stirring is followed by the separation of the liquid extract and the solid. Separation can be accomplished by decantation, centrifugation, filtration, or any other process separating solids from the liquid that is known to those who are skilled in the art.

Step 2: The two extracts from Step 2 were combined and concentrated to about 400 mL by distillation, evaporation, or similar method. Preferably, the concentration occurs under vacuum at ambient temperature to avoid degradation of the desired proanthocyanidin compounds. Acetone solvent is recovered and recycled for use within the process. If desired, the acetone can be purified by filtration, distillation, and the like before reuse. The amount of solid in the extract was approximately 130 grams. The obtained organic extract contains a mixture of cinnamon components.

Step 3: The concentrated acetone extract is mixed with an appropriate volume of water (about 800-1200 mL). The mixture is then subjected to the removal of acetone by evaporation or distillation. The removal of acetone is preferably accomplished in vacuo to avoid elevated temperatures, which may have an adverse effect on the oligomeric proanthocyanidins. When the acetone is removed and the remaining solution is aqueous, the hydrophobic components from the extract precipitate in water in a form of oil drops, gum or other semi-solids or solids. Many of these insoluble hydrophobic compounds are the same which are responsible for the aroma of cinnamon. The desired proanthocyanidins remain dissolved in the aqueous phase.

Step 5: After the removal of acetone, the precipitated in water hydrophobic components of extract can be removed by filtering, decantation, or centrifugation. Another way to remove the hydrophobic components is to wash the aqueous mixture with an organic solvent, preferably comprising or consisting essentially of a hexane-ethyl acetate (1:1 v/v) or other solvent or solvent mixtures that dissolves the hydrophobic components (among them there are the components responsible for the characteristic aroma of cinnamon bark) but not the proanthocyanidins. It may be necessary to repeat the wash with another portion of hexane-ethyl acetate (prepared at the ratio of 1:1, v/v). It is important that the organic wash should have a polarity suitable to dissolve the hydrophobic components of cinnamon bark, but it should not be polar enough to dissolve proanthocyanins. This solvent wash removes the oily extract components and in addition helps to remove precipitated reddish polymeric proanthocyanidins. After the organic wash process, the desired proanthocyanidins remain dissolved in the aqueous phase.

Step 6: The clear aqueous phases from Step 5 is then evaporated to dryness, preferably in vacuo in order to avoid thermal degradation of the proanthocyanidins. The solid obtained from this evaporation (28 g) is rich in proanthocyanidins and is largely devoid of the compounds that are less polar and of those that are more polar than proanthocyanidins.

Step 7: Further enhancement of the concentration of proanthocyanidins can be obtained by treating the solid with a small volume of dry acetone. Proanthocyanidins dissolve in acetone, while about 20% w/w of the initial weight has poor solubility in dry acetone and remains largely insoluble. After removal of the undesired solids either by decantation, filtration or centrifugation, the clear acetone solution contains oligomeric proanthocyanidins. The recovery of the proanthocyanidins from the acetone solution can be done preferably by the evaporation of the acetone in vacuo.

The yield of the final extract is usually between 4 and 7% of the weight of the cinnamon bark, which is higher than conventional water extraction processes. The yield of the final solid is 22 g (4.4 wt %) based on total weight of the starting cinnamon bark extract.

What is claimed is:

1. A process for recovering oligomeric proanthocyanidins from a source of proanthocyanidins that comprises:
a. contacting a source of catechin type-A proanthocyanidins under extraction conditions with a first organic solvent to form a first extract and spent solids;
b. combining the first extract with a volume of water to form an aqueous-organic solvent solution;
c. removing first organic solvent from the aqueous-organic solvent solution to form a first aqueous solution containing soluble proanthocyanidins and to separate at least a portion of the hydrophobic components from said first aqueous solution in the form of oil droplets, oily clumps, or other solids;
d. in one or more stages, washing the first aqueous solution with a second organic solvent that dissolves residual hydrophobic components present in the first aqueous solution under conditions sufficient to remove additional water insoluble components and form a second aqueous solution; and
e. exposing the second aqueous solution to evaporation conditions until a solid product containing oligomeric proanthocyanidins is obtained.

2. A process according to claim 1 wherein said dry solid contains oligomeric catechin type-A proanthocyanidins.

3. A process according to claim 2 further comprising
f. further contacting said solid product with a third organic solvent to form an extract phase containing catechin type-A proanthocyanidins and an insoluble residue;
g. recovering catechin type-A proanthocyanidins from said purified liquid extract phase.

4. A process according to claim 1 wherein the first and third organic solvents each comprise acetone.

5. A process according to claim 1 wherein the first extract is combined with 200-400 vol % water to form said liquid-liquid solution.

6. A process according to claim 1 wherein said first extract is concentrated by evaporation or distillation before water is added thereto in step b.

7. A process according to claim 6 wherein said first extract is concentrated to a dissolved solids concentration within the range of 1-99 wt %.

8. A process according to claim 1 wherein said second organic solvent comprises hexane and ethyl acetate.

9. A process according to claim 8 wherein said second organic solvent exhibits a weight ratio of 1 part hexane to 0.1-10 parts ethyl acetate.

10. A process according to claim 1 wherein compounds responsible for a cinnamon aroma are soluble in said second organic solvent.

11. A preparation comprising oligomeric proanthocyanidins recovered from a source of proanthocyanidins by a process that comprises the steps:
a. contacting a source of catechin type-A proanthocyanidins under extraction conditions with a first organic solvent to form a first extract and spent solids;
b. combining the first extract with a volume of water to form an aqueous-organic solvent solution;
c. removing first organic solvent from the aqueous-organic solvent solution to form a first aqueous solution containing soluble proanthocyanidins and to separate at least a portion of the hydrophobic components from said first aqueous solution in the form of oil droplets, oily clumps, or other solids;
d. in one or more stages, washing the first aqueous solution with a second organic solvent that dissolves residual hydrophobic components present in the first aqueous solution under conditions sufficient to remove additional water insoluble components and form a second aqueous solution; and
e. exposing the second aqueous solution to evaporation conditions until a solid product containing oligomeric proanthocyanidins is obtained.
